(19) United States
(12) Patent Application Publication
(10) Pub. No.: US 2008/0176836 A1
(43) Pub. Date: Jul. 24, 2008

(54) Use of selected CGRP antagonists for combating menopausal hot flushes

(76) Inventors: Klaus Rudolf, Warthausen (DE); Henri Doods, Warthausen (DE); Stephan Georg Mueller, Warthausen (DE); Annette Zamponi, Biberach (DE); Philipp Lustenberger, Warthausen (DE); Kirsten Arndt, Biberach (DE); Gerhard Schaenzle, Biberach-Mettenberg (DE); Dirk Stenkamp, Biberach (DE); Rolf-Stefan Brickl, Warthausen (DE)

Correspondence Address: MICHAEL P. MORRIS;BOEHRINGER INGELHEIM USA CORPORATION, 900 RIDGEBURY RD, P. O. BOX 368, RIDGEFIELD, CT, 06877-0368, US

(21) Appl. No.: 11/774,980

(22) Filed: Jul. 9, 2007

Related U.S. Application Data
(63) Continuation of application No. 11/301,446, filed on Dec. 13, 2005, now abandoned.

(30) Foreign Application Priority Data
Dec. 29, 2004 (DE) 102004063752

(51) Int. Cl. A61K 31/551

(52) U.S. Cl. 514/221

(57) ABSTRACT

The invention relates to the use of selected CGRP antagonists, the physiologically acceptable salts thereof or the hydrates of the hydrates of the salts thereof for combating menopausal hot flushes.

### BACKGROUND TO THE INVENTION

Hot flushes (also called hot flashes) are a common symptom of peri/post-menopausal syndrome, the physiology of which is still not completely understood to this day. Apart from hormone replacement therapy, which constitutes a complex intervention and frequently cannot be used long-term on account of its side effects, there is at present no simple therapy with few side effects for this generally problematic manifestation.

Hot flushes are caused by vasodilatation and increased blood flow. It has already been speculated in the literature on causation numerous occasions that CGRP (calcitonin gene-related peptide) plays a part in the production of menopausal hot flushes in oestrogen-deficient women on account of the vasodilatory properties of this neuropeptide ([1]: J. Endocrinol. (1995), 146(3), 431-437; [2]: Acta Physiol. Scand. (1998), 162(4), 517-522; [3]: Am. J. Obstet. Gynecol. (1996), 175(3, Pt. 1), 638-642). The therapeutic use of CGRP antagonists for treating menopausal syndrome has not hitherto been suggested in the literature.

It has now been found that the symptoms of menopausal hot flushes can be effectively prevented, or their effects can be significantly lessened, by substances which antagonise the effects of CGRP (CGRP antagonists), and this therapeutic approach is particularly distinguished from hormone replacement by the absence of side effects.

The present invention thus relates to the use of selected CGRP antagonists, the physiologically acceptable salts thereof or the hydrates of the salts thereof, for combating menopausal hot flushes, including both prevention and acute treatment. The novel use preferably relates to monotherapy with a single substance, but also includes combined therapy with a plurality of substances from the above-mentioned category of active substances. In addition, the use according to the invention may also be carried out in addition to conventional hormone replacement therapy.

The invention further relates to the use of selected CGRP antagonists, the physiologically acceptable salts thereof or the hydrates of the salts thereof, for preparing a pharmaceutical composition for combating menopausal hot flushes, as well as the corresponding pharmaceutical compositions containing as active substance one or more of the selected CGRP antagonists, the physiologically acceptable salts thereof or the hydrates of the salts thereof.

The following compounds are preferred examples of CGRP antagonists for combating menopausal hot flushes, for preparing a corresponding pharmaceutical composition and as an ingredient of a corresponding pharmaceutical composition:
(1) 4-(2-oxo-1,2,4,5-tetrahydro-1,3-benzdiazepin-3-yl)-piperidine-1-carboxylic acid-{(R)-1-(4-amino-3-chloro-5-ethyl-benzyl)-2-[4-(4-methyl-piperazin-1-yl)-piperidin-1-yl]-2-oxo-ethyl}-amide,

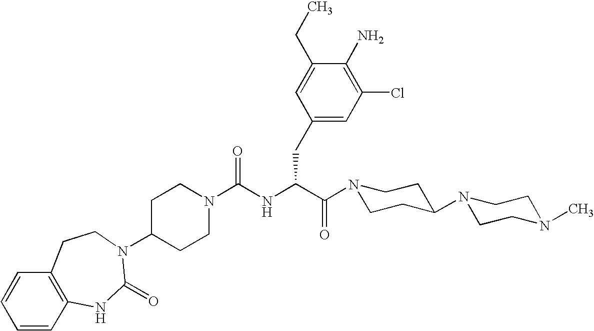

(2) [1′-((R)-3-(4-amino-3-chloro-5-trifluoromethyl-phenyl)-2-{[4-(2-oxo-1,2,4,5-tetrahydro-1,3-benzdiazepin-3-yl)-piperidine-1-carbonyl]-amino}-propionyl)-4,4′-bipiperidinyl-1-yl]-acetic acid,

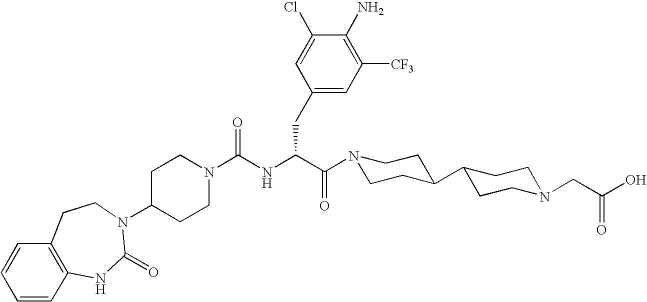

(3) 3-{1-[(R)-1-(4-amino-3,5-dibromo-benzyl)-2-[1,4′]bipiperidinyl-1′-yl-2-oxo-ethyl carbamoyl]-piperidin-4-yl}-2-oxo-1,2,3,4-tetrahydro-quinazoline-7-carboxylic acid,

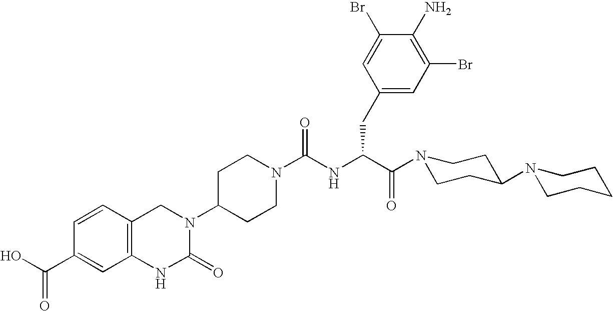

(4) (R)-1-(7-methyl-1H-benzotriazol-5-ylmethyl)-2-[4-(4-methyl-piperazin-1-yl)-piperidin-1-yl]-2-oxo-ethyl 4-(2-oxo-1,2,4,5-tetrahydro-1,3-benzodiazepin-3-yl)-piperidine-1-carboxylate,

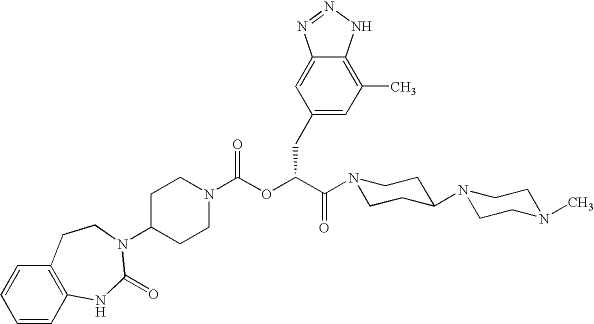

(5) (S)-2-(3-chloro-4-hydroxy-5-trifluoromethyl-benzyl)-1-[4-(1-methyl-piperi-din-4-yl)-piperazin-1-yl]-4-[4-(2-oxo-1,2,4,5-tetrahydro-1,3-benzodiazepin-3-yl)-piperidin-1-yl]-butan-1,4-dione,

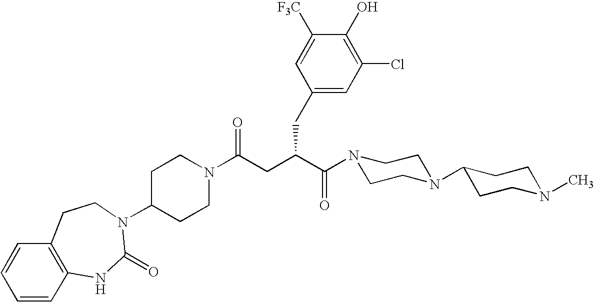

(6) (R)-1-(4-hydroxy-3,5-dimethyl-benzyl)-2-oxo-2-(4-piperidin-4-yl-piperazin-1-yl)-ethyl 4-(2-oxo-1,2,4,5-tetrahydro-1,3-benzodiazepin-3-yl)-piperidine-1-carboxylate,

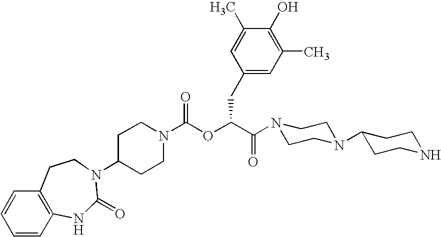

(7) (R)-1-(3,5-dibromo-4-hydroxy-benzyl)-2-[4-(1-methyl-piperidin-4-yl)-piperazin-1-yl]-2-oxo-ethyl 4-(2-oxo-1,2,4,5-tetrahydro-1,3-benzodiazepin-3-yl)-piperidine-1-carboxylate,

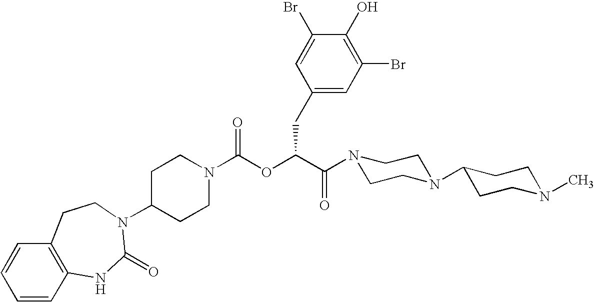

(8) (R)-1-(6-amino-5-methyl-pyridin-3-ylmethyl)-2-oxo-2-(4-piperazin-1-yl-piperidin-1-yl)-ethyl 4-(2-oxo-1,2,4,5-tetrahydro-1,3-benzodiazepin-3-yl)-piperidine-1-carboxylate,

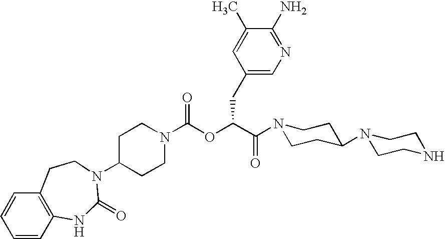

(9) (R)-1-(3,5-dibromo-4-hydroxy-benzyl)-2-oxo-2-(4-piperazin-1-yl-piperidin-1-yl)-ethyl 4-(2-oxo-1,2,4,5-tetrahydro-1,3-benzodiazepin-3-yl)-piperidine-1-carboxylate,

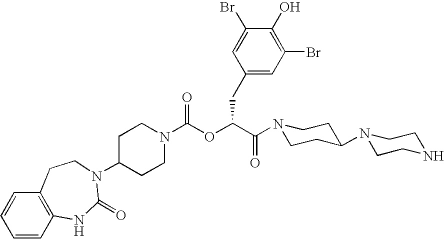

(10) (R)-1-(3,5-dibromo-4-hydroxy-benzyl)-2-oxo-2-(4-piperidin-4-yl-piperazin-1-yl)-ethyl 4-(2-oxo-1,2,4,5-tetrahydro-1,3-benzodiazepin-3-yl)-piperidine-1-carboxylate,

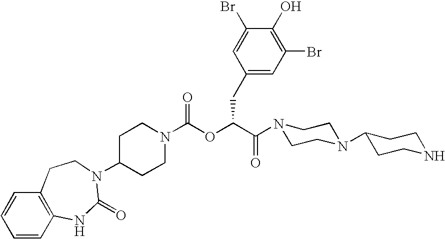

(11) (S)-2-(4-amino-3-chloro-5-trifluoromethyl-benzyl)-1-[4-(4-methyl-piperazin-1-yl)-piperidin-1-yl]-4-[4-(2-oxo-1,2,4,5-tetrahydro-1,3-benzodiazepin-3-yl)-piperidin-1-yl]-butan-1,4-dione,

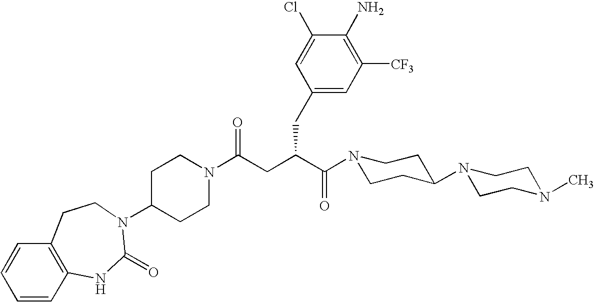

(12) 4-(2-oxo-1,2,4,5-tetrahydro-1,3-benzodiazepin-3-yl)-piperidine-1-carboxylic acid-{(R)-1-(3,4-diethyl-benzyl)-2-[4-(1-methyl-piperidin-4-yl)-piperazin-1-yl]-2-oxo-ethyl}-amide,

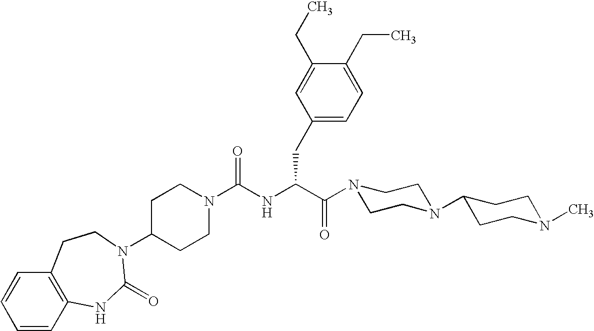

(13) (R)-1-(4-amino-3-chloro-5-trifluoromethyl-benzyl)-2-[4-(4-methyl-piperazin-1-yl)-piperidin-1-yl]-2-oxo-ethyl 4-(2-oxo-1,2,4,5-tetrahydro-1,3-benzodiazepin-3-yl)-piperidine-1-carboxylate,

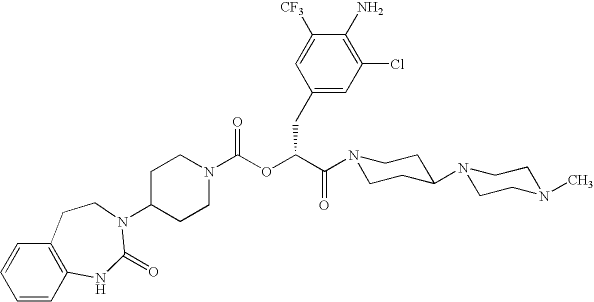

(14) 4-(2-oxo-1,2,4,5-tetrahydro-1,3-benzodiazepin-3-yl)-piperidine-1-carboxylic acid-{(R)-1-(4-amino-3-chloro-5-trifluoromethyl-benzyl)-2-[4-(4-methyl-piperazin-1-yl)-piperidin-1-yl]-2-oxo-ethyl}-amide,

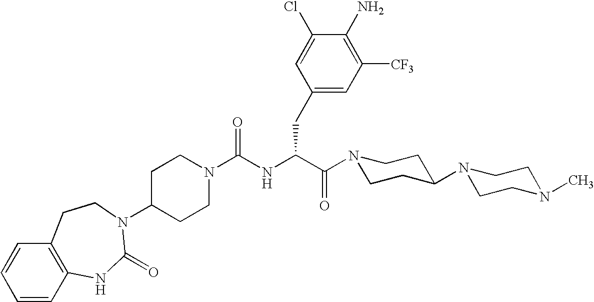

(15) (S)-2-(4-amino-3,5-bis-trifluoromethyl-benzyl)-1-[4-(4-methyl-piperazin-1-yl)-piperidin-1-yl]-4-[4-(2-oxo-1,2,4,5-tetrahydro-1,3-benzodiazepin-3-yl)-piperidin-1-yl]-butan-1,4-dione,

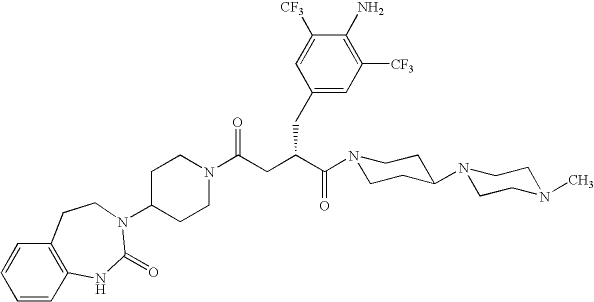

(16) 4-(2-oxo-1,2,4,5-tetrahydro-1,3-benzodiazepin-3-yl)-piperidine-1-carboxylic acid-{(R)-1-(4-amino-3,5-bis-trifluoromethyl-benzyl)-2-[4-(4-methyl-piperazin-1-yl)-piperidin-1-yl]-2-oxo-ethyl}-amide,

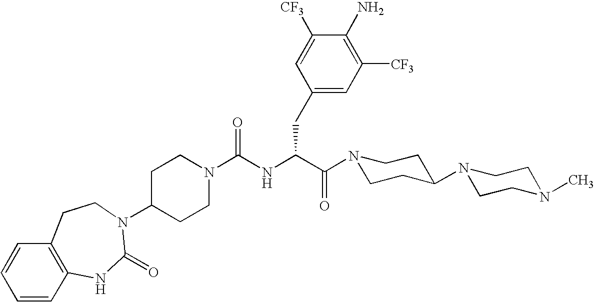

(17) (R)-1-(4-amino-3,5-bis-trifluoromethyl-benzyl)-2-[4-(4-methyl-piperazin-1-yl)-piperidin-1-yl]-2-oxo-ethyl 4-(2-oxo-1,2,4,5-tetrahydro-1,3-benzodiazepin-3-yl)-piperidine-1-carboxylate,

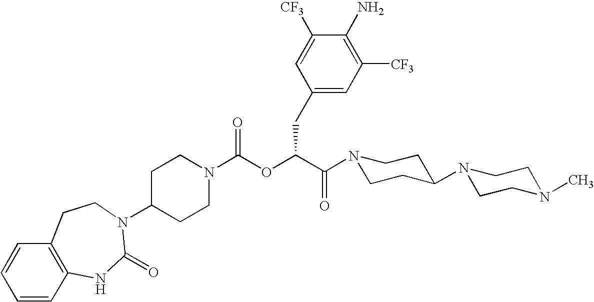

(18) 4-(2-oxo-1,2,4,5-tetrahydro-1,3-benzodiazepin-3-yl)-piperidine-1-carboxylic acid-{(R)-1-(4-amino-3-chloro-5-methyl-benzyl)-2-[4-(1-methyl-piperidin-4-yl)-piperazin-1-yl]-2-oxo-ethyl}-amide,

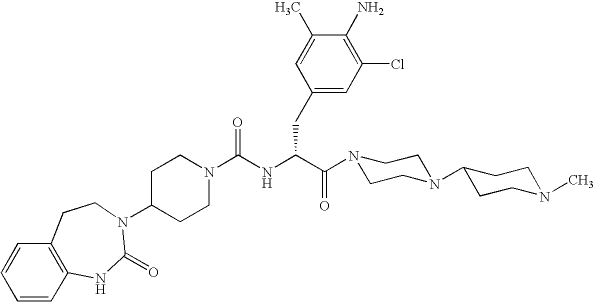

(19) (R)-1-(3,5-dibromo-4-hydroxy-benzyl)-2-[4-(4-methyl-piperazin-1-yl)-piperidin-1-yl]-2-oxo-ethyl 4-(2-oxo-1,2,4,5-tetrahydro-1,3-benzodiazepin-3-yl)-piperidine-1-carboxylate,

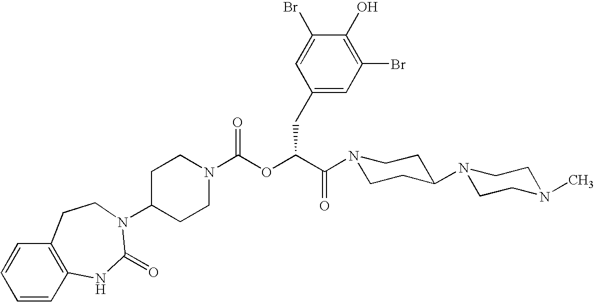

(20) (R)-1-(4-hydroxy-3,5-dimethyl-benzyl)-2-oxo-2-(4-piperazin-1-yl-piperidin-1-yl)-ethyl 4-(2-oxo-1,2,4,5-tetrahydro-1,3-benzodiazepin-3-yl)-piperidine-1-carboxylate,

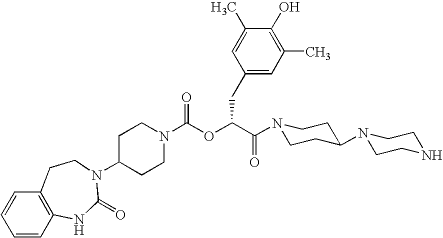

(21) (R)-1-(4-hydroxy-3,5-dimethyl-benzyl)-2-[4-(1-methyl-piperidin-4-yl)-piperazin-1-yl]-2-oxo-ethyl 4-(2-oxo-1,2,4,5-tetrahydro-1,3-benzodiazepin-3-yl)-piperidine-1-carboxylate,

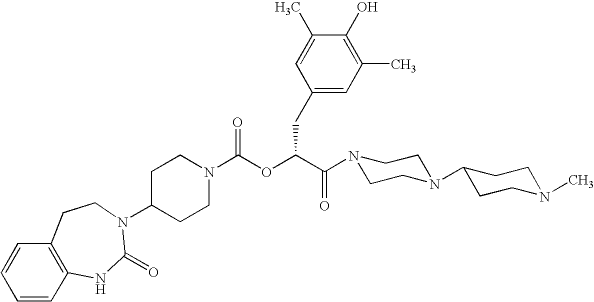

(22) (S)-1-1,4′-bipiperidinyl-1′-yl-2-(3-chloro-4-hydroxy-5-trifluoromethyl-benzyl)-4-[4-(2-oxo-1,2,4,5-tetrahydro-1,3-benzodiazepin-3-yl)-piperidin-1-yl]-butan-1,4-dione,

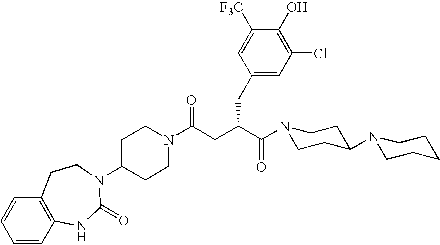

a physiologically acceptable salt thereof or one of the hydrates thereof.

The dosage needed to achieve the desired effect is expediently 0.0001 to 3 mg/kg of body weight, preferably 0.01 to 1 mg/kg of body weight, when administered by intravenous or subcutaneous route, and 0.01 to 20 mg/kg of body weight, preferably 0.1 to 20 mg/kg of body weight when administered orally, and 0.01 to 10 mg/kg of body weight, preferably 0.1 to 10 mg/kg of body weight, when administered by nasal route or by inhalation, one to three times a day in each case.

If the treatment with the selected CGRP antagonists is being given in addition to conventional hormone replacement therapy, it is advisable to reduce the dosages specified above, the dosage then being from 1/5 of the lower limits specified above to 1/1 of the upper limits specified above.

For this purpose the selected CGRP antagonists, the physiologically acceptable salts thereof or the hydrates of the salts thereof may be formulated together with one or more inert conventional carriers and/or diluents, e.g. with maize starch, lactose, glucose, microcrystalline cellulose, magnesium stearate, polyvinylpyrrolidone, citric acid, tartaric acid, water, water/ethanol, water/glycerol, water/sorbitol, water/polyethyleneglycol, propyleneglycol, cetylstearylalcohol, carboxymethylcellulose or fatty substances such as hard fat or suitable mixtures thereof, into conventional galenic preparations such as tablets, coated tablets, capsules, powders, suspensions, solutions, metered dose aerosols or suppositories.

Possible pharmaceutical preparations are illustrated below:

capsules for powder inhalation containing 1 mg active substance,
inhalable solution for nebuliser containing 1 mg active substance,
propellant gas-operated metered dose aerosol containing 1 mg active substance,
nasal spray containing 1 mg active substance,
tablets containing 20 mg active substance,
capsules containing 20 mg active substance,
aqueous solution for nasal application containing 10 mg active substance,
aqueous solution for nasal application containing 5 mg active substance, or
suspension for nasal application containing 20 mg active substance.

CGRP is released by sensory nerves, for example by the trigeminal nerve which innervates half the skin of the face. It has already been shown that irritation of the trigeminal ganglion in humans leads to an increase in the CGRP plasma level and causes reddening of the face ([4]: P. J. Goadsby et al., Annals of Neurology, Vol. 23, No. 2, 1988, 193-196).

The Examples that follow describe pharmaceutical forms for application which contain as active substance one of the selected CGRP antagonists for use according to the invention.

The selected CGRP antagonists (A) may be administered for example using one of the following pharmaceutical formulations:

capsules for powder inhalation, containing 0.1 to 50 mg, preferably 0.3 to 30 mg of (A);
nasal spray containing 2 to 50 mg, preferably 5 to 40 mg of (A);
tablets containing 10 to 600 mg, preferably 30 to 400 mg of (A);
pellets for capsules containing varying parts by weight of (A);
extruded materials for capsules or tablets containing varying parts by weight of (A);
suppositories containing 50 to 600 mg, preferably 30 to 400 mg of (A);
injectable solutions containing 0.2 to 30 mg, preferably 0.5 to 15 mg of (A);

The following Examples describe pharmaceutical preparations containing as active substance one of the selected CGRP antagonists according to the invention, a physiologically acceptable salt thereof or a hydrate of the salt. To begin with, a Table is provided in which the pharmaceutical components have been allocated numbers which serve to identify the active substances in the following tables of Examples.

### Pharmaceutical Components

| Substance no. | substance |
|---|---|
| 1 | CGRP antagonist (1) or a physiologically acceptable salt |
| | thereof [1a] |
| 2 | CGRP antagonist (2) or a physiologically acceptable salt |
| | thereof [2a] |
| 3 | CGRP antagonist (3) or a physiologically acceptable salt |
| | thereof [3a] |
| 4 | CGRP antagonist (4) or a physiologically acceptable salt |
| | thereof [4a] |
| 5 | CGRP antagonist (5) or a physiologically acceptable salt |
| | thereof [5a] |
| 6 | CGRP antagonist (6) or a physiologically acceptable salt |
| | thereof [6a] |
| 7 | CGRP antagonist (7) or a physiologically acceptable salt |
| | thereof [7a] |
| 8 | CGRP antagonist (8) or a physiologically acceptable salt |
| | thereof [8a] |
| 9 | CGRP antagonist (9) or a physiologically acceptable salt |
| | thereof [9a] |
| 10 | CGRP antagonist (10) or a physiologically acceptable |
| | salt thereof [10a] |
| 11 | CGRP antagonist (11) or a physiologically acceptable |
| | salt thereof [11a] |
| 12 | CGRP antagonist (12) or a physiologically acceptable |
| | salt thereof [12a] |
| 13 | CGRP antagonist (13) or a physiologically acceptable |
| | salt thereof [13a] |
| 14 | CGRP antagonist (14) or a physiologically acceptable |
| | salt thereof [14a] |
| 15 | CGRP antagonist (15) or a physiologically acceptable |
| | salt thereof [15a] |
| 16 | CGRP antagonist (16) or a physiologically acceptable |
| | salt thereof [16a] |
| 17 | CGRP antagonist (17) or a physiologically acceptable |
| | salt thereof [17a] |
| 18 | CGRP antagonist (18) or a physiologically acceptable |
| | salt thereof [18a] |
| 19 | CGRP antagonist (19) or a physiologically acceptable |
| | salt thereof [19a] |
| 20 | CGRP antagonist (20) or a physiologically acceptable |
| | salt thereof [20a] |
| 21 | CGRP antagonist (21) or a physiologically acceptable |
| | salt thereof [21a] |
| 22 | CGRP antagonist (22) or a physiologically acceptable |
| | salt thereof [22a] |

### EXAMPLE 1a

### Tablets Containing 100 Mg CGRP Antagonist

Composition/tablet:

| | | |
|---|---|---|
| CGRP antagonist | 100 | mg |
| lactose | 375 | mg |
| magnesium stearate | 3.0 | mg |
| povidone | 8.5 | mg |
| crospovidone | 14.4 | mg |

volatile ingredient: water

### Preparation:

CGRP antagonist and lactose (fine) are homogeneously mixed in a suitable mixer (e.g. Diosna P2); then the mixture is granulated with an aqueous povidone solution; the granulated material is screened with a Kressner screen of mesh size 1.6 mm and dried for 2 hours at 40° C. Then the granulated material is screened in a suitable mill, e.g. a Comill at 3000 rpm with a mesh size of 1.1 mm. Then the granulated material is mixed for 5 minutes with crospovidone and then for another 1 minute with magnesium stearate. The mixture thus obtained is compressed in a tablet press to form tablets of the required diameter.

### EXAMPLE 1b

### Tablets Containing 10 Mg CGRP Antagonist

Composition/tablet:

| | | |
|---|---|---|
| CGRP antagonist | 10 | mg |
| lactose | 475 | mg |
| magnesium stearate | 3.0 | mg |
| povidone | 8.5 | mg |
| crospovidone | 14.4 | mg |

volatile ingredient: water

### Preparation:

CGRP antagonist and lactose (fine) are homogeneously mixed in a suitable mixer (e.g. Diosna P2); then the mixture is granulated with an aqueous povidone solution; the granulated material is screened with a 1.6 mm Kressner screen and dried for 2 hours at 40° C. Then the granulated material is screened in a suitable mill, e.g. a Comill at 3000 rpm with a mesh size of 1.1 mm. Then the granulated material is mixed for 5 minutes with crospovidone and then for another 1 minute with magnesium stearate. The mixture thus obtained is compressed in a tablet press to form tablets of the required diameter.

### EXAMPLE 1c

### Tablets Containing 600 Mg CGRP Antagonist

Composition/tablet:

| | |
|---|---|
| CGRP antagonist | 600 mg |
| lactose | 175 mg |
| magnesium stearate | 6 mg |
| povidone | 17 mg |
| crospovidone | 28.8 mg |

volatile ingredient: water

### Preparation:

CGRP antagonist and lactose (fine) are homogeneously mixed in a suitable mixer (e.g. Diosna P2); then the mixture is granulated with an aqueous povidone solution; the granulated material is screened with a 1.6 mm Kressner screen and dried for 2 hours at 40° C. Then the granulated material is screened in a suitable mill, e.g. a Comill at 3000 rpm with a mesh size of 1.1 mm. Then the granulated material is mixed for 5 minutes with crospovidone and then for another 1 minute with magnesium stearate. The mixture thus obtained is compressed in a tablet press to form tablets of the required diameter.

### EXAMPLE 1d

### Tablets Containing 100 Mg CGRP Antagonist

Composition/tablet:

| | |
|---|---|
| CGRP antagonist | 100 mg |
| lactose | 403 mg |
| magnesium stearate | 3.1 mg |
| povidone | 9.1 mg |
| crospovidone | 15.3 mg |

volatile ingredient: water

### Preparation:

CGRP antagonist and lactose (fine) are homogeneously mixed in a suitable mixer (e.g. Diosna P2); then the mixture is granulated with an aqueous povidone solution; the granulated material is screened with a 1.6 mm Kressner screen and dried for 2 hours at 40° C. Then the granulated material is screened in a suitable mill, e.g. a Comill at 3000 rpm with a mesh size of 1.1 mm. Then the granulated material is mixed for 5 minutes with crospovidone and then for another 1 minute with magnesium stearate. The mixture thus obtained is compressed in a tablet press to form tablets of the required diameter. These methods of preparation are the basis for further Examples listed in the following Table.

### Table Relating to Examples 1a-d

| | | | | | | mg | |
|---|---|---|---|---|---|---|---|
| | substance | | mg | mg | mg | magnesium | |
| Ex. | no. | mg | lactose | povidone | crospovidone | stearate | ø [mm] |
| 1.1 | 13 | 40 | 80.0 | 1.8 | 3.0 | 0.6 | 7 |
| 1.2 | 6a | 100 | 200.0 | 4.5 | 7.6 | 1.6 | 9 |
| 1.3 | 1a | 70 | 140.0 | 3.2 | 5.3 | 1.1 | 8 |
| 1.4 | 22 | 180 | 360.0 | 8.1 | 13.7 | 2.8 | 12 |
| 1.5 | 6 | 120 | 240.0 | 5.4 | 9.1 | 1.9 | 10 |
| 1.6 | 3 | 10 | 70.0 | 1.2 | 2.0 | 0.4 | 6 |
| 1.7 | 17 | 270 | 540.0 | 12.2 | 20.6 | 4.2 | 13 |
| 1.8 | 3a | 220 | 440.0 | 9.9 | 16.7 | 3.4 | 13 |
| 1.9 | 14 | 140 | 280.0 | 6.3 | 10.7 | 2.2 | 11 |
| 1.10 | 5 | 230 | 460.0 | 10.4 | 17.5 | 3.6 | 13 |
| 1.11 | 21 | 230 | 460.0 | 10.4 | 17.5 | 3.6 | 13 |
| 1.12 | 6 | 40 | 80.0 | 1.8 | 3.0 | 0.6 | 7 |
| 1.13 | 3 | 80 | 160.0 | 3.6 | 6.1 | 1.2 | 9 |
| 1.14 | 4a | 320 | 540.0 | 12.9 | 21.8 | 4.5 | 13 |
| 1.15 | 13 | 340 | 580.0 | 13.8 | 23.3 | 4.8 | 13 |
| 1.16 | 2 | 170 | 340.0 | 7.7 | 12.9 | 2.7 | 12 |
| 1.17 | 21a | 110 | 220.0 | 5.0 | 8.4 | 1.7 | 11 |
| 1.18 | 5 | 170 | 340.0 | 7.7 | 12.9 | 2.7 | 12 |
| 1.19 | 5a | 320 | 540.0 | 12.9 | 21.8 | 4.5 | 13 |
| 1.20 | 14 | 30 | 60.0 | 1.4 | 2.3 | 0.5 | 6 |
| 1.21 | 2a | 600 | 600.0 | 18.0 | 30.5 | 6.2 | 13 |
| 1.22 | 5 | 300 | 600.0 | 13.5 | 22.8 | 4.7 | 13 |
| 1.23 | 7 | 160 | 320.0 | 7.2 | 12.2 | 2.5 | 12 |
| 1.24 | 17a | 160 | 320.0 | 7.2 | 12.2 | 2.5 | 12 |
| 1.25 | 4 | 170 | 340.0 | 7.7 | 12.9 | 2.7 | 12 |

### EXAMPLE 2a

### Tablets Containing 100 Mg CGRP Antagonist

Composition:

| | |
|---|---|
| CGRP antagonist | 100 mg |
| lactose | 284 mg |
| microcrystalline cellulose | 89.5 mg |
| magnesium stearate | 7.2 mg |
| croscarmellose | 7.3 mg |

volatile ingredient: water

### Preparation:

CGRP antagonist, lactose (fine) and microcrystalline cellulose are homogeneously mixed in a suitable mixer (e.g. Diosna P2); then the mixture is granulated with water. The granulated material is screened with a 1.6 mm Kressner screen and dried for 2 hours at 40° C. Then the granulated material is screened in a suitable mill, e.g. a Comill at 3000 rpm with a mesh size of 1.1 mm. Then the granulated material is mixed for 5 minutes with croscarmellose and then for another 1 minute with magnesium stearate. The mixture thus obtained is compressed in a tablet press to form tablets of the required diameter.

### EXAMPLE 2b

### Tablets Containing 10 Mg CGRP Antagonist

Composition:

| | |
|---|---|
| CGRP antagonist | 10 mg |
| lactose | 274 mg |
| microcrystalline cellulose | 109.5 mg |
| magnesium stearate | 7.2 mg |
| croscarmellose | 7.3 mg |

volatile ingredient: water

### Preparation:

CGRP antagonist, lactose (fine) and microcrystalline cellulose are homogeneously mixed in a suitable mixer (e.g. Diosna P2); then the mixture is granulated with water. The granulated material is screened with a 1.6 mm Kressner screen and dried for 2 hours at 40° C. Then the granulated material is screened in a suitable mill, e.g. a Comill at 3000 rpm with a mesh size of 1.1 mm. Then the granulated material is mixed for 5 minutes with croscarmellose and then for another 1 minute with magnesium stearate. The mixture thus obtained is compressed in a tablet press to form tablets of the required diameter.

### EXAMPLE 2c

### Tablets Containing 400 Mg CGRP Antagonist

Composition:

| | |
|---|---|
| CGRP antagonist | 400 mg |
| lactose | 194 mg |
| microcrystalline cellulose | 95 mg |
| magnesium stearate | 7.2 mg |
| croscarmellose | 7.3 mg |

volatile ingredient: water

### Preparation:

CGRP antagonist, lactose (fine) and microcrystalline cellulose are homogeneously mixed in a suitable mixer (e.g. Diosna P2); then the mixture is granulated with water. The granulated material is screened with a 1.6 mm Kressner screen and dried for 2 hours at 40° C. Then the granulated material is screened in a suitable mill, e.g. a Comill at 3000 rpm with a mesh size of 1.1 mm. Then the granulated material is mixed for 5 minutes with croscarmellose and then for another 1 minute with magnesium stearate. The mixture thus obtained is compressed in a tablet press to form tablets of the required diameter.

### EXAMPLE 2d

### Tablets Containing 100 Mg CGRP Antagonist

Composition:

| | |
|---|---|
| CGRP antagonist | 100 mg |
| lactose | 403 mg |
| microcrystalline cellulose | 121 mg |
| magnesium stearate | 9.3 mg |
| croscarmellose | 9.4 mg |

volatile ingredient: water

### Preparation:

CGRP antagonist, lactose (fine) and microcrystalline cellulose are homogeneously mixed in a suitable mixer (e.g. Diosna P2); then the mixture is granulated with water. The granulated material is screened with a 1.6 mm Kressner screen and dried for 2 hours at 40° C. The dry granulated material is screened in a suitable mill, e.g. a Comill at 3000 rpm with a mesh size of 1.1 mm. Then the granulated material is mixed for 5 minutes with croscarmellose and then for another 1 minute with magnesium stearate. The mixture thus obtained is compressed in a tablet press to form tablets of the required diameter.

These methods of preparation are the basis for further Examples which are listed in the following Table.

### Table Relating to Example 2a-d

| | | | | mg | | | |
|---|---|---|---|---|---|---|---|
| | substance | | mg | microcryst. | mg Mg- | mg cros- | |
| Example | no. | mg | lactose | cellulose | stearate | carmellose | ø [mm] |
| 2.1 | 5 | 130 | 195.0 | 6.5 | 5.0 | 5.0 | 10 |
| 2.2 | 4a | 380 | 570.0 | 19.0 | 14.5 | 14.8 | 13 |
| 2.3 | 6 | 150 | 225.0 | 7.5 | 5.7 | 5.8 | 10 |
| 2.4 | 6a | 240 | 360.0 | 12.0 | 9.2 | 9.3 | 12 |
| 2.5 | 16 | 30 | 45.0 | 1.5 | 1.1 | 1.2 | 6 |
| 2.6 | 3 | 600 | 400.0 | 20.0 | 15.3 | 15.5 | 13 |
| 2.7 | 2a | 220 | 330.0 | 11.0 | 8.4 | 8.5 | 12 |
| 2.8 | 2 | 30 | 45.0 | 1.5 | 1.1 | 1.2 | 6 |
| 2.9 | 14 | 120 | 180.0 | 6.0 | 4.6 | 4.7 | 9 |
| 2.10 | 12 | 40 | 60.0 | 2.0 | 1.5 | 1.6 | 6 |
| 2.11 | 21 | 110 | 165.0 | 5.5 | 4.2 | 4.3 | 9 |
| 2.12 | 5a | 180 | 270.0 | 9.0 | 6.9 | 7.0 | 12 |
| 2.13 | 6 | 310 | 465.0 | 15.5 | 11.9 | 12.0 | 13 |
| 2.14 | 13 | 390 | 585.0 | 19.5 | 14.9 | 15.1 | 13 |
| 2.15 | 1a | 10 | 150.0 | 3.2 | 2.4 | 2.5 | 8 |
| 2.16 | 5 | 240 | 360.0 | 12.0 | 9.2 | 9.3 | 13 |
| 2.17 | 17 | 50 | 75.0 | 2.5 | 1.9 | 1.9 | 7 |
| 2.18 | 3 | 90 | 135.0 | 4.5 | 3.4 | 3.5 | 8 |
| 2.19 | 7 | 190 | 285.0 | 9.5 | 7.3 | 7.4 | 12 |
| 2.20 | 6 | 360 | 540.0 | 18.0 | 13.8 | 14.0 | 13 |

### EXAMPLE 3a

### Aqueous Solution for Intranasal Application Containing 20% CGRP Antagonist

Composition:

| | | |
|---|---|---|
| CGRP antagonist | 20 | mg |
| mannitol | 5 | mg |
| water | ad 0.1 | ml |

### Method:

The active substance is dissolved in water with stirring and optionally heating. The isotonic agent mannitol is added and the solution is made up to the final volume with water.

### EXAMPLE 3b

### Aqueous Solution for Intranasal Application Containing 2% CGRP Antagonist

Composition:

| | | |
|---|---|---|
| CGRP antagonist | 2 | mg |
| mannitol | 5 | mg |
| water | ad 0.1 | ml |

### Method:

The active substance is in dissolved in water with stirring and optionally heating. The isotonic agent mannitol is added and the solution is made up to the final volume with water.

### EXAMPLE 3c

### Aqueous solution for intranasal application containing 40% CGRP antagonist

Composition:

| | | |
|---|---|---|
| CGRP antagonist | 40 | mg |
| mannitol | 5 | mg |
| water | ad 0.1 | ml |

### Method:

The active substance is dissolved in water with stirring and optionally heating. The isotonic agent mannitol is added and the solution is made up to the final volume with water.

### EXAMPLE 3d

### Aqueous Solution for Intranasal Application Containing 20% CGRP Antagonist and 1.5% Labrasol

Composition:

| | | |
|---|---|---|
| CGRP antagonist | 20 | mg |
| Labrasol | 1.5 | mg |
| mannitol | 5 | mg |
| water | ad 0.1 | ml |

### Method:

The active substance is dissolved in water with stirring and optionally heating. The isotonic agent mannitol and Labrasol are added and the solution is made up to the final volume with water.

### EXAMPLE 3e

### Aqueous Solution for Intranasal Application Containing 50% CGRP Antagonist and 1.5% Labrasol

Composition:

| | | |
|---|---|---|
| CGRP antagonist | 50 | mg |
| Labrasol | 1.5 | mg |
| mannitol | 5 | mg |
| water | ad 0.1 | ml |

### Method:

The active substance is dissolved in water with stirring and optionally heating. The isotonic agent mannitol and Labrasol are added and the solution is made up to the final volume with water.

These methods of preparation are the basis for further Examples which are listed in the following Table.

### Table Relating to Example 3a-e

| | CGRP | | | |
|---|---|---|---|---|
| | antagonist | | | |
| Example | no. | mg | mg mannitol | mg Labrasol |
| 3.1 | 13 | 20 | 5 | 3.00 |
| 3.2 | 22 | 10 | 5 | 1.50 |
| 3.3 | 1a | 10 | 5 | 3.00 |
| 3.4 | 5a | 20 | 5 | 1.50 |
| 3.5 | 6 | 10 | 5 | 0.00 |
| 3.6 | 13 | 5 | 5 | 1.50 |
| 3.7 | 4a | 10 | 5 | 3.00 |
| 3.8 | 3a | 5 | 5 | 3.00 |
| 3.9 | 3 | 20 | 5 | 3.00 |
| 3.10 | 1 | 5 | 5 | 0.00 |
| 3.11 | 7 | 10 | 5 | 1.50 |
| 3.12 | 12 | 10 | 5 | 3.00 |
| 3.13 | 4 | 20 | 5 | 3.00 |
| 3.14 | 2 | 5 | 5 | 0.00 |
| 3.15 | 14 | 20 | 5 | 0.00 |

### Pellets

The pharmaceutical substances according to the invention may also be prepared in the form of small particles such as pellets, for example. The active substance may be applied to neutral pellets consisting of sucrose and starch or microcrystalline cellulose. If acidic or basic excipients make it easier for an active substance to dissolve, on account of the active substance having a pH-dependent solubility, it is also possible to use acid or basic starter cores instead of neutral pellets. The preparation comprises the following steps:

1. selection or preparation of starter pellets
2. formation of the layer of active substance
Optional: coating pellets to improve their stability or correct the flavour or—if desired—delay the release of one or more active substances.

### EXAMPLE 4a

### Preparation of Basic Starter Cores

Composition:

| | |
|---|---|
| Povidone K25 | 3 parts by weight |
| Microcryst. cellulose | 20 parts by weight |
| Meglumin | 77 parts by weight |

77 parts by weight meglumin, 20 parts by weight microcryst. cellulose and 3 parts by weight Povidone K25 are mixed for 15 minutes in a gyro wheel mixer. Then the powder mixture is placed in a twin-screw extruder at a rate of approx. 1 kg/h together with water which is added using a metering pump. The metering of the water is automatically regulated so as to obtain a rated torque of approx. 19% in the extruder. The extrusion is carried out through a nozzle plate drilled with holes 0.8 mm in diameter.

The extruded strips are rounded off into pellets in a spheronizer, the rounding operation lasting for approx. 3 minutes at approx. 850 RPM.

The pellets are dried at 80° C. for approx. 1.5 hours in a fluidised bed dryer.

The core material is fractionated through a tumbler screening machine with different perforated bases with nominal mesh sizes of 0.71 to 1.25 mm. The fractions of material of between 0.71 and 0.90 and 0.90 and 1.12 mm which are appropriate in each case are used in the later processes.

### EXAMPLE 4b

### Preparation of an Application of Active Substance Containing 100 Mg CGRP Antagonist

Composition:

| | |
|---|---|
| core material | 200 parts by weight |
| hydroxypropylcellulose | 38 parts by weight |
| talc | 20 parts by weight |
| CGRP antagonist | 100 parts by weight |

Hydroxypropylcellulose is dissolved with stirring in 250 parts by weight of 2-propanol and then the active substance and talc are dispersed in this solution with stirring. In a fluidised bed processing apparatus 200 parts by weight of core material are sprayed with the dispersion containing the active substance at an air entry temperature of 20° to 30° C. by the under-bed spraying method. The pellets containing the active substance are then dried in the circulating air dryer at 35° C. for 8 hours.

To remove lumps, the pellets containing the active substance are screened using a screen with a nominal mesh size of 1.25 mm. The fraction of material (particle size<1.25 mm) is processed further.

The active substance layer is generally built up in the same way every time, but the type and amount of active substance, the nature and quantity of the binder, the amount of talc and the amounts of water, isopropanol and ethanol vary.

### EXAMPLE 4c

### Preparation of an Application of Active Substance Containing 10 Parts by Weight of CGRP Antagonist

Composition:

| | |
|---|---|
| core material | 100 parts by weight |
| hydroxypropylcellulose | 24 parts by weight |
| talc | 12 parts by weight |
| CGRP antagonist | 10 parts by weight |

Hydroxypropylcellulose is dissolved with stirring in 250 parts by weight of 2-propanol and then the active substance and talc are dispersed in this solution with stirring. In a fluidised bed processing apparatus 200 parts by weight of core material are sprayed with the dispersion containing the active substance at an air entry temperature of 20° to 30° C. by the under-bed spraying method. The pellets containing the active substance are then dried in the circulating air dryer at 35° C. for 8 hours.

To remove lumps, the pellets containing the active substance are screened using a screen with a nominal mesh size of 1.25 mm. The fraction of material (particle size<1.25 mm) is processed further.

### EXAMPLE 4d

### Preparation of an Application of Active Substance Containing 400 Parts by Weight of CGRP Antagonist

Composition:

| | |
|---|---|
| core material | 100 parts by weight |
| hydroxypropylcellulose | 62 parts by weight |
| talc | 24 parts by weight |
| CGRP antagonist | 400 parts by weight |

Hydroxypropylcellulose is dissolved with stirring in 250 parts by weight of 2-propanol and then the active substance and talc are dispersed in this solution with stirring. In a fluidised bed processing apparatus 100 parts by weight of core material are sprayed with the dispersion containing the active substance at an air entry temperature of 20° to 30° C. by the under-bed spraying method. The pellets containing the active substance are then dried in the circulating air dryer at 35° C. for 8 hours.

To remove lumps, the pellets containing the active substance are screened using a screen with a nominal mesh size of 1.25 mm. The fraction of material (particle size<1.25 mm) is processed further.

The active substance layer is generally built up in the same way every time, but the type and amount of active substance, the nature and quantity of the binder, the amount of talc and the amounts of water, isopropanol and ethanol vary.

The respective amounts may vary and are shown in tabulated form hereinafter.

The Examples contain 10 to 380 parts by weight of CGRP antagonist either as an active form, in the form of a physiologically acceptable salt or in the form of the hydrate of a salt, while the rest of the composition is shown in the following Table.

### Table Relating to Example 4b-d

| | CGRP | | | | *pbw | | *pbw | | |
|---|---|---|---|---|---|---|---|---|---|
| | antagonist | | *pbw | *pbw | starter | *pbw | iso- | *pbw | *pbw |
| Ex. | no. | pbw | povidone | HPC | pellets | talc | propanol | ethanol | water |
| 4.1 | 22 | 70 | 14.0 | 0.0 | 70.0 | 15.4 | 2630 | 0 | 0 |
| 4.2 | 2 | 240 | 48.0 | 0.0 | 240.0 | 52.8 | 1600 | 0 | 1600 |
| 4.3 | 15a | 60 | 0.0 | 12.0 | 60.0 | 13.2 | 0 | 1600 | 0 |
| 4.4 | 1a | 230 | 0.0 | 46.0 | 230.0 | 50.6 | 0 | 0 | 1770 |
| 4.5 | 1 | 40 | 0.0 | 8.0 | 450.0 | 49.8 | 4210 | 0 | 0 |
| 4.6 | 2 | 220 | 0.0 | 44.0 | 220.0 | 48.4 | 0 | 0 | 2940 |
| 4.7 | 15 | 380 | 76.0 | 0.0 | 380.0 | 83.6 | 3610 | 0 | 0 |
| 4.8 | 7 | 380 | 0.0 | 76.0 | 380.0 | 83.6 | 2230 | 0 | 0 |
| 4.9 | 4 | 230 | 0.0 | 46.0 | 230.0 | 50.6 | 0 | 1640 | 0 |
| 4.10 | 21a | 360 | 72.0 | 0.0 | 360.0 | 79.2 | 1700 | 0 | 0 |
| 4.11 | 6 | 250 | 0.0 | 50.0 | 250.0 | 55.0 | 0 | 0 | 1760 |
| 4.12 | 4 | 280 | 0.0 | 56.0 | 280.0 | 61.6 | 0 | 1800 | 0 |
| 4.13 | 3 | 360 | 72.0 | 0.0 | 360.0 | 79.2 | 0 | 2400 | 0 |
| 4.14 | 14 | 120 | 0.0 | 24.0 | 360.0 | 50.4 | 0 | 0 | 4950 |
| 4.15 | 4a | 310 | 0.0 | 62.0 | 310.0 | 68.2 | 0 | 0 | 2670 |
| 4.16 | 6 | 600 | 0.0 | 120.0 | 600.0 | 132.0 | 1900 | 0 | 0 |
| 4.17 | 12 | 280 | 56.0 | 0.0 | 280.0 | 61.6 | 2230 | 0 | 0 |
| 4.18 | 7 | 350 | 70.0 | 0.0 | 350.0 | 77.0 | 0 | 1610 | 0 |
| 4.19 | 5 | 10 | 2.0 | 0.0 | 100.0 | 11.2 | 0 | 0 | 1930 |
| 4.20 | 3 | 180 | 0.0 | 36.0 | 180.0 | 39.6 | 1870 | 0 | 0 |
| 4.21 | 4 | 100 | 20.0 | 0.0 | 100.0 | 22.0 | 0 | 1680 | 0 |
| 4.22 | 16 | 80 | 16.0 | 0.0 | 80.0 | 17.6 | 1900 | 0 | 0 |
| 4.23 | 4 | 20 | 0.0 | 4.0 | 350.0 | 37.4 | 0 | 0 | 1930 |
| 4.24 | 6a | 300 | 0.0 | 60.0 | 300.0 | 66.0 | 0 | 2890 | 0 |
| 4.25 | 2 | 290 | 0.0 | 58.0 | 290.0 | 63.8 | 2670 | 0 | 0 |
| 4.26 | 12 | 280 | 56.0 | 0.0 | 280.0 | 61.6 | 1890 | 0 | 0 |
| 4.27 | 3a | 70 | 14.0 | 0.0 | 70.0 | 15.4 | 0 | 3210 | 0 |
| 4.28 | 14a | 50 | 0.0 | 10.0 | 50.0 | 11.0 | 0 | 0 | 2890 |
| 4.29 | 7a | 40 | 8.0 | 0.0 | 140.0 | 18.8 | 2600 | 0 | 0 |

*pbw = parts by weight

### EXAMPLE 4e

### Delaying the Release of the Pellets Containing the Active Substance

### The Active Substance Pellets Contain One of the Active Substances 1-22

Composition:

| | |
|---|---|
| pellets containing active substance | 30 parts by weight |
| Eudragit S 100 | 4 parts by weight |
| Eudragit RS 100 | 2 parts by weight |
| triethylcitrate | 1.25 parts by weight |
| hydroxypropylcellulose | 0.61 parts by weight |
| talc | 0.25 parts by weight |

4 parts by weight Eudragit S100, 2 parts by weight Eudragit RS100, 1.25 parts by weight triethylcitrate and 0.61 parts by weight hydroxypropylcellulose are dissolved In 112 parts by weight of 96% ethanol with stirring. Then 0.25 parts by weight of talc are dispersed in the solution with stirring.

In a fluidised bed processing apparatus 30 parts by weight of pellets containing active substance are sprayed with the delayed-release dispersion at an air entry temperature of 35° C. to 40° C. by the under-bed spraying method.

The isolated core material is then dried in the circulating air dryer at 40° C. for 8 hours. To remove lumps, the dried delayed-release pellets are screened using a screen with a nominal mesh size of 1.5 mm. The fraction of material (particle size<1.5 mm) is processed further.

A summary of the various delayed-release coatings is given in Table 4f.

TABLE 4F
The numbers correspond to parts by weight

Example

| | 4.30 | 4.31 | 4.32 | 4.33 |
|---|---|---|---|---|
| pellets of active substance | 30 | 30 | 30 | 30 |
| ethylcellulose | 4 | 6 | | |
| polyethyleneglycol | 0.5 | 0.4 | | |
| Eudragit S100 | | | 3 | 5 |
| Eudragit RS100 | | | 3 | 1 |
| triethylcitrate | | | 1.25 | 1.25 |
| hydroxypropylcellulose | | | 0.61 | 0.61 |
| talc | 1.2 | 1.0 | 0.25 | 0.25 |

### Extruded Materials

The pharmaceutical substances according to the invention may also be prepared in the form of extruded materials which after being cut up or spheronised are packed directly into capsules or ground up and then made into tablets.

The preparation comprises the following steps:

### 1. Extrusion

2a. Cutting up/spheronising
2b. Grinding and then processing to form tablets

### EXAMPLE 5a

### Preparation of Wet Extruded Materials

Composition:

| | |
|---|---|
| Povidone K25 | 6 parts by weight |
| microcrystalline cellulose | 40 parts by weight |
| CGRP antagonist | 100 parts by weight |

100 parts by weight of CGRP antagonist, 40 parts by weight microcrystalline cellulose (Avicel PH 101) and 6 parts by weight of povidone (Collidone K25) are mixed for 15 minutes in a gyro wheel mixer. Then the powder mixture is placed in a twin-screw extruder at a rate of approx. 1 kg/h together with water which is added using a metering pump. The metering of the water is automatically regulated so as to obtain a rated torque of approx. 19% in the extruder. The extrusion is carried out through a nozzle plate drilled with holes 0.8 mm in diameter.

The extruded strips are rounded off into pellets in a spheronizer, the rounding operation lasting for approx. 3 minutes at about 850 rpm.

The pellets are dried at 80° C. for approx. 1.5 hours in a fluidised bed dryer.

The core material is fractionated through a tumbler screening machine with different perforated bases with nominal mesh sizes of 0.71 to 1.25 mm. The fractions of material of between 0.71 and 0.90 and 0.90 and 1.12 mm which are appropriate in each case are used in the later processes.

### EXAMPLE 5b

### Preparation of Wet Extruded Material

Composition:

| | |
|---|---|
| povidone K25 | 4 parts by weight |
| microcrystalline cellulose | 30 parts by weight |
| CGRP antagonist | 10 parts by weight |

100 parts by weight of CGRP antagonist, 30 parts by weight microcrystalline cellulose (Avicel PH 101) and 4 parts by weight of povidone (Collidone K25) are mixed for 15 minutes in a gyrowheel mixer. Then the powder mixture is placed in a twin-screw extruder at a rate of approx. 1 kg/h together with water which is added using a metering pump. The metering of the water is automatically regulated so as to obtain a rated torque of approx. 19% in the extruder. The extrusion is carried out through a nozzle plate drilled with holes 0.8 mm in diameter.

The extruded strips are rounded off into pellets in a spheronizer, the rounding operation lasting for approx. 3 minutes at about 850 rpm.

The pellets are dried at 80° C. for approx. 1.5 hours in a fluidised bed dryer.

The core material is fractionated through a tumbler screening machine with different perforated bases with nominal mesh sizes of 0.71 to 1.25 mm. The fractions of material of between 0.71 and 0.90 and 0.90 and 1.12 mm which are appropriate in each case are used in the later processes.

### EXAMPLE 5c

### Preparation of wet extruded material

Composition:

| | |
|---|---|
| povidone K25 | 15 parts by weight |
| microcrystalline cellulose | 110 parts by weight |
| CGRP antagonist | 400 parts by weight |

400 parts by weight of CGRP antagonist, 110 parts by weight microcrystalline cellulose (Avicel PH 101) and 15 parts by weight of povidone (Collidone K25) are mixed for 15 minutes in a gyrowheel mixer. Then the powder mixture is placed in a twin-screw extruder at a rate of approx. 1 kg/h together with water which is added using a metering pump. The metering of the water is automatically regulated so as to obtain a rated torque of approx. 19% in the extruder. The extrusion is carried out through a nozzle plate drilled with holes 0.8 mm in diameter.

The extruded strips are rounded off into pellets in a spheronizer, the rounding operation lasting for approx. 3 minutes at about 850 rpm.

The pellets are dried at 80° C. for approx. 1.5 hours in a fluidised bed dryer.

The core material is fractionated through a tumbler screening machine with different perforated bases with nominal mesh sizes of 0.71 to 1.25 mm. The fractions of material of between 0.71 and 0.90 and 0.90 and 1.12 mm which are appropriate in each case are used in the later processes.

This preparation method is the basis for further combined examples which are listed in the following Table.

### Table Relating to Example 5a-c

| | CGRP | | pbw | pbw | polyethylene- |
|---|---|---|---|---|---|
| Ex. | antagonist no. | pbw | povidone | poloxamer | glycol 4000 |
| 5.1 | 21 | 80 | 28.0 | 2.7 | 84 |
| 5.2 | 1a | 110 | 38.5 | 3.7 | |
| 5.3 | 14a | 170 | 59.5 | 5.7 | |
| 5.4 | 6a | 100 | 35.0 | 3.4 | |
| 5.5 | 15 | 80 | 28.0 | 2.7 | |
| 5.6 | 6 | 20 | 7.0 | 0.7 | 21 |
| 5.7 | 5 | 200 | 70.0 | 6.8 | 210 |
| 5.8 | 2a | 40 | 14.0 | 1.4 | 42 |
| 5.9 | 7 | 50 | 17.5 | 1.7 | 52.5 |
| 5.10 | 17 | 70 | 24.5 | 2.4 | 73.5 |
| 5.11 | 2 | 110 | 38.5 | 3.7 | |
| 5.12 | 22 | 600 | 210.0 | 20.3 | |
| 5.13 | 1 | 130 | 45.5 | 4.4 | |
| 5.14 | 5a | 40 | 14.0 | 1.4 | 42 |
| 5.15 | 1 | 160 | 56.0 | 5.4 | |
| 5.16 | 13 | 60 | 21.0 | 2.0 | 63 |
| 5.17 | 4 | 200 | 70.0 | 6.8 | |
| 5.18 | 6 | 80 | 28.0 | 2.7 | 84 |
| 5.19 | 16 | 150 | 52.5 | 5.1 | |
| 5.20 | 3 | 10 | 3.5 | 0.3 | 10.5 |

*pbw = parts by weight

### EXAMPLE 6a

### Preparation of Molten Extruded Material

Composition:

| | |
|---|---|
| povidone K25 | 6 parts by weight |
| poloxamer | 40 parts by weight |
| CGRP antagonist | 100 parts by weight |

100 parts by weight CGRP antagonist, 40 parts by weight poloxamer and 6 parts by weight povidone K25 are mixed for 15 minutes in a gyrowheel mixer. Then the powder mixture is placed in a twin-screw extruder at a rate of approx. 1 kg/h together with water which is added using a metering pump. The temperature is regulated so as to obtain a rated torque of approx. 19% in the extruder. The extrusion is carried out through a nozzle plate drilled with holes 0.8 mm in diameter.

The extruded strips emerging are cut by chopping off the top, and the extruded strips are rounded off into pellets in a spheronizer, the rounding operation lasting for approx. 3 minutes at about 850 rpm.

The pellets are dried at 80° C. for approx. 1.5 hours in a fluidised bed dryer.

The core material is fractionated through a tumbler screening machine with different perforated bases with nominal mesh sizes of 0.71 to 1.25 mm. The fractions of material of between 0.71 and 0.90 and 0.90 and 1.12 mm which are appropriate in each case are used in the later processes.

### EXAMPLE 6b

### Preparation of Molten Extruded Material

Composition:

| | |
|---|---|
| povidone K25 | 2 parts by weight |
| poloxamer | 30 parts by weight |
| CGRP antagonist | 10 parts by weight |

10 parts by weight CGRP antagonist, 30 parts by weight poloxamer and 2 parts by weight povidone K25 are mixed for 15 minutes in a gyrowheel mixer. Then the powder mixture is placed in a twin-screw extruder at a rate of approx. 1 kg/h together with water which is added using a metering pump. The temperature is regulated so as to obtain a rated torque of approx. 19% in the extruder. The extrusion is carried out through a nozzle plate drilled with holes 0.8 mm in diameter.

The extruded strips emerging are cut by chopping off the top, and the extruded strips are rounded off into pellets in a spheronizer, the rounding operation lasting for approx. 3 minutes at about 850 rpm at about 40° C.

The pellets are dried at 80° C. for approx. 1.5 hours in a fluidised bed dryer.

The core material is fractionated through a tumbler screening machine with different perforated bases with nominal mesh sizes of 0.71 to 1.25 mm. The fractions of material of between 0.71 and 0.90 and 0.90 and 1.12 mm which are appropriate in each case are used in the later processes.

### EXAMPLE 6c

### Preparation of Molten Extruded Material

Composition:

| | |
|---|---|
| povidone K25 | 18 parts by weight |
| poloxamer | 132 parts by weight |
| CGRP antagonist | 400 parts by weight |

400 parts by weight CGRP antagonist, 132 parts by weight poloxamer and 18 parts by weight povidone K25 are mixed for 15 minutes in a gyrowheel mixer. Then the powder mixture is placed in a twin-screw extruder at a rate of approx. 1 kg/h together with water which is added using a metering pump. The temperature is regulated so as to obtain a rated torque of approx. 19% in the extruder. The extrusion is carried out through a nozzle plate drilled with holes 0.8 mm in diameter.

The extruded strips emerging are cut by chopping off the top, and the extruded strips are rounded off into pellets in a spheronizer, the rounding operation lasting for approx. 3 minutes at about 850 rpm at about 40° C.

The pellets are dried at 80° C. for approx. 1.5 hours in a fluidised bed dryer.

The core material is fractionated through a tumbler screening machine with different perforated bases with nominal mesh sizes of 0.71 to 1.25 mm. The fractions of material of between 0.71 and 0.90 and 0.90 and 1.12 mm which are appropriate in each case are used in the later processes.

The different compositions may vary, and further Examples are given below in the form of a Table.

### Table relating to Example 6a-c

| | CGRP | | pbw | pbw | polyethylene- |
|---|---|---|---|---|---|
| Ex. | antagonist no. | pbw | povidone | poloxamer | glycol 4000 |
| 6.1 | 7a | 120 | 6.0 | 31.5 | |
| 6.2 | 7 | 130 | 6.5 | 34.1 | |
| 6.3 | 12a | 90 | 4.5 | 23.6 | 70.88 |
| 6.4 | 3a | 40 | 2.0 | 10.5 | 31.50 |
| 6.5 | 6a | 30 | 1.5 | 7.9 | 23.63 |
| 6.6 | 2 | 20 | 1.0 | 5.3 | 15.75 |
| 6.7 | 16 | 110 | 5.5 | 28.9 | |
| 6.8 | 5a | 180 | 9.0 | 47.3 | |
| 6.9 | 11a | 150 | 7.5 | 39.4 | |
| 6.1 | 3 | 90 | 4.5 | 23.6 | |
| 6.11 | 6 | 190 | 9.5 | 49.9 | |
| 6.12 | 13 | 600 | 30.0 | 157.5 | |
| 6.13 | 15 | 130 | 6.5 | 34.1 | |
| 6.14 | 5 | 150 | 7.5 | 39.4 | |
| 6.15 | 1 | 130 | 6.5 | 34.1 | |
| 6.16 | 4a | 110 | 5.5 | 28.9 | 86.63 |
| 6.17 | 4 | 180 | 9.0 | 47.3 | |
| 6.18 | 5 | 90 | 4.5 | 23.6 | |
| 6.19 | 7 | 150 | 7.5 | 39.4 | |
| 6.20 | 14 | 100 | 5.0 | 26.3 | |
| 6.21 | 1a | 70 | 3.5 | 18.4 | 55.13 |
| 6.22 | 21 | 20 | 1.0 | 5.3 | 15.75 |
| 6.23 | 4 | 200 | 10.0 | 52.5 | |
| 6.24 | 3 | 10 | 0.5 | 2.6 | 7.88 |
| 6.25 | 22 | 30 | 1.5 | 7.9 | 23.63 |

*pbw = parts by weight

### EXAMPLE 7

### Further Processing to Form Tablets

The extruded materials are ground up in a suitable mill and the resulting granulated material is further processed with conventional tabletting excipients analogously to Example 1 to produce tablets.

### Powder Inhalant

### Preparation of Spherically Nanostructured Microparticles of the Active Substances for Preparing a Powder Inhalant

The active substances are dissolved in an ethanol/water (4:1) mixture in order to prepare a 4 wt. % active substance solution and the active substance solution is sprayed so as to produce a spray mist with a droplet size having the characteristic value X50 (median value=the particle size/droplet size below which 50% of the quantity of particles falls, with respect to the volume distribution of the individual particles/drops) in the range from 1.5 to 8 μm, and wherein Q(5.8) (corresponds to the amount of particles below 5.8 μm, based on the volume distribution of the droplets) is between 30% and 100%. The spray mist thus obtained is dried using a drying gas with an entry temperature of between 130° C. and 200° C. and an exit temperature of 40° C. to 120° C. The flow volume of the spray gas is 1 Nm³/h to 15 Nm³/h and the flow volume of the drying gas is 15 Nm³/h to 150 Nm³/h. The dried solid fraction is collected using a gravity separator and/or filter unit.

### EXAMPLE 8

### Capsules for Powder Inhalation Containing 0.5 Mg Active Substance

Composition:
1 capsule for powder inhalation contains:

| | |
|---|---|
| CGRP antagonist | 0.5 mg |
| lactose | 20 mg |
| hard gelatine capsules | 50 mg |

### Preparation Method:

The CGRP antagonist is prepared as spherically nanostructured active substance particles and homogeneously mixed with lactose. The mixture is packed into hard gelatine capsules.

### Table relating to Example 8

| Example | CGRP antagonist no. | mg | mg lactose |
|---|---|---|---|
| 8.1 | 4 | 30.00 | 80.00 |
| 8.2 | 22 | 10.00 | 60.00 |
| 8.3 | 1 | 20.00 | 70.00 |
| 8.4 | 16 | 30.00 | 80.00 |
| 8.5 | 6 | 25.00 | 75.00 |
| 8.6 | 1 | 30.00 | 80.00 |
| 8.7 | 3 | 20.00 | 70.00 |
| 8.8 | 1 | 10.00 | 60.00 |
| 8.9 | 13 | 20.00 | 70.00 |
| 8.10 | 1 | 0.30 | 50.30 |
| 8.11 | 5 | 0.10 | 50.10 |
| 8.12 | 15 | 30.00 | 80.00 |
| 8.13 | 6 | 30.00 | 80.00 |
| 8.14 | 21 | 3.00 | 53.00 |
| 8.15 | 2 | 20.00 | 70.00 |
| 8.16 | 5 | 5.00 | 55.00 |
| 8.17 | 16 | 20.00 | 70.00 |
| 8.18 | 2 | 10.00 | 60.00 |
| 8.19 | 4 | 10.00 | 60.00 |
| 8.20 | 14 | 0.00 | 50.00 |
| 8.21 | 6 | 10.00 | 60.00 |
| 8.22 | 3 | 15.00 | 65.00 |
| 8.23 | 4 | 10.00 | 60.00 |
| 8.24 | 14 | 50.00 | 100.00 |
| 8.25 | 6 | 30.00 | 80.00 |
| 8.26 | 5 | 0.00 | 50.00 |
| 8.27 | 12 | 20.00 | 70.00 |

### EXAMPLE 9

### Injectable Solution Containing 0.5 Mg CGRP Antagonist

Composition:

| | |
|---|---|
| CGRP antagonist | 0.5 mg |
| physiological saline solution | |

The active substance is dissolved in physiological saline solution.

The dosage amounts may vary and are shown hereinafter in table form.

The Examples contain 0.2 to 30 mg CGRP antagonist.

### Table Relating to Example 9

| Example | CGRP antagonist no. | mg |
|---|---|---|
| 9.1 | 5 | 0.20 |
| 9.2 | 14a | 14.30 |
| 9.3 | 6 | 4.40 |
| 9.4 | 6a | 10.30 |
| 9.5 | 16 | 1.80 |
| 9.6 | 3 | 1.30 |
| 9.7 | 2a | 4.40 |
| 9.8 | 22 | 9.40 |
| 9.9 | 4 | 2.60 |
| 9.10 | 2 | 8.20 |
| 9.11 | 1 | 4.30 |
| 9.12 | 15a | 25.50 |
| 9.13 | 6 | 14.20 |
| 9.14 | 21 | 13.40 |
| 9.15 | 1a | 5.40 |
| 9.16 | 5 | 6.90 |

### EXAMPLE 10

### Suppositories containing 200 mg CGRP antagonist

Composition:

| | | |
|---|---|---|
| CGRP antagonist | 200 | mg |
| hard wax | ad 2 | g |

### Preparation:

The hard wax is melted and the active substance is suspended in the mass. Then the mass is poured into suitable suppository moulds.

The dosage amounts may vary and are shown hereinafter in table form. The Examples contain 50 to 600 mg of CGRP antagonist.

### Table Relating to Example 10

| Example | CGRP antagonist no. | mg |
|---|---|---|
| 1.1 | 3 | 250 |
| 1.2 | 6a | 150 |
| 1.3 | 21a | 460 |
| 1.4 | 2 | 540 |
| 1.5 | 16 | 320 |
| 1.6 | 3 | 180 |
| 1.7 | 7 | 150 |
| 1.8 | 3a | 480 |
| 1.9 | 14 | 600 |
| 1.10 | 5 | 180 |

What is claimed is:

1. A method for treating for combating menopausal hot flushes which comprises administering to a human female suffering from the same a therapeutically effective amount of a compound selected from the group consisting of:
(1) 4-(2-oxo-1,2,4,5-tetrahydro-1,3-benzodiazepin-3-yl)-piperidine-1-carboxylic acid-{(R)-1-(4-amino-3-chloro-5-ethyl-benzyl)-2-[4-(4-methyl-piperazin-1-yl)-piperidin-1-yl]-2-oxo-ethyl}-amide,
(2) [1′-((R)-3-(4-amino-3-chloro-5-trifluoromethyl-phenyl)-2-{[4-(2-oxo-1,2,4,5-tetra-hydro-1,3-benzodiazepin-3-yl)-piperidine-1-carbonyl]-amino}-propionyl)-4,4′-bi-piperidinyl-1-yl]-acetic acid,
(3) 3-{1-[(R)-1-(4-amino-3,5-dibromo-benzyl)-2-[1,4′]bipiperidinyl-1′-yl-2-oxo-ethyl-carbamoyl]-piperidin-4-yl}-2-oxo-1,2,3,4-tetrahydro-quinazoline-7-carboxylic acid,
(4) (R)-1-(7-methyl-1H-benzotriazol-5-ylmethyl)-2-[4-(4-methyl-piperazin-1-yl)-piperidin-1-yl]-2-oxo-ethyl 4-(2-oxo-1,2,4,5-tetrahydro-1,3-benzodiazepin-3-yl)-piperidine-1-carboxylate,
(5) (S)-2-(3-chloro-4-hydroxy-5-trifluoromethyl-benzyl)-1-[4-(1-methyl-piperidin-4-yl)-piperazin-1-yl]-4-[4-(2-oxo-1,2,4,5-tetrahydro-1,3-benzodiazepin-3-yl)-piperidin-1-yl]-butan-1,4-dione,
(6) (R) —-(4-hydroxy-3,5-dimethyl-benzyl)-2-oxo-2-(4-piperidin-4-yl-piperazin-1-yl)-ethyl 4-(2-oxo-1,2,4,5-tetrahydro-1,3-benzodiazepin-3-yl)-piperidine-1-carboxylate,
(7) (R)-1-(3,5-dibromo-4-hydroxy-benzyl)-2-[4-(1-methyl-piperidin-4-yl)-piperazin-1-yl]-2-oxo-ethyl 4-(2-oxo-1,2,4,5-tetrahydro-1,3-benzodiazepin-3-yl)-piperidine-1-carboxylate,
(8) (R)-1-(6-amino-5-methyl-pyridin-3-ylmethyl)-2-oxo-2-(4-piperazin-1-yl-piperidin-1-yl)-ethyl 4-(2-oxo-1,2,4,5-tetrahydro-1,3-benzodiazepin-3-yl)-piperidine-1-carboxylate,
(9) (R)-1-(3,5-dibromo-4-hydroxy-benzyl)-2-oxo-2-(4-piperazin-1-yl-piperidin-1-yl)-ethyl 4-(2-oxo-1,2,4,5-tetrahydro-1,3-benzodiazepin-3-yl)-piperidine-1-carboxylate,
(10) (R)-1-(3,5-dibromo-4-hydroxy-benzyl)-2-oxo-2-(4-piperidin-4-yl-piperazin-1-yl)-ethyl 4-(2-oxo-1,2,4,5-tetrahydro-1,3-benzodiazepin-3-yl)-piperidine-1-carboxylate,
(11) (S)-2-(4-amino-3-chloro-5-trifluoromethyl-benzyl)-1-[4-(4-methyl-piperazin-1-yl)-piperidin-1-yl]-4-[4-(2-oxo-1,2,4,5-tetrahydro-1,3-benzodiazepin-3-yl)-piperidin-1-yl]-butan-1,4-dione,
(12) 4-(2-oxo-1,2,4,5-tetrahydro-1,3-benzodiazepin-3-yl)-piperidine-1-carboxylic acid-{(R)-1-(3,4-diethyl-benzyl)-2-[4-(1-methyl-piperidin-4-yl)-piperazin-1-yl]-2-oxo-ethyl}-amide,
(13) (R) —-(4-amino-3-chloro-5-trifluoromethyl-benzyl)-2-[4-(4-methyl-piperazin-1-yl)-piperidin-1-yl]-2-oxo-ethyl 4-(2-oxo-1,2,4,5-tetrahydro-1,3-benzodiazepin-3-yl)-piperidine-1-carboxylate,
(14) 4-(2-oxo-1,2,4,5-tetrahydro-1,3-benzodiazepin-3-yl)-piperidine-1-carboxylic acid-{(R)-1-(4-amino-3-chloro-5-trifluoromethyl-benzyl)-2-[4-(4-methyl-piperazin-1-yl)-piperidin-1-yl]-2-oxo-ethyl}-amide,
(15) (S)-2-(4-amino-3,5-bis-trifluoromethyl-benzyl)-1-[4-(4-methyl-piperazin-1-yl)-piperidin-1-yl]-4-[4-(2-oxo-1,2,4,5-tetrahydro-1,3-benzodiazepin-3-yl)-piperidin-1-yl]-butan-1,4-dione,
(16) 4-(2-oxo-1,2,4,5-tetrahydro-1,3-benzodiazepin-3-yl)-piperidine-1-carboxylic acid-{(R)-1-(4-amino-3,5-bis-trifluoromethyl-benzyl)-2-[4-(4-methyl-piperazin-1-yl)-piperidin-1-yl]-2-oxo-ethyl}-amide,
(17) (R)-1-(4-amino-3,5-bis-trifluoromethyl-benzyl)-2-[4-(4-methyl-piperazin-1-yl)-piperidin-1-yl]-2-oxo-ethyl 4-(2-oxo-1,2,4,5-tetrahydro-1,3-benzodiazepin-3-yl)-piperidine-1-carboxylate,
(18) 4-(2-oxo-1,2,4,5-tetrahydro-1,3-benzodiazepin-3-yl)-piperidine-1-carboxylic acid-{(R)-1-(4-amino-3-chloro-5-methyl-benzyl)-2-[4-(1-methyl-piperidin-4-yl)-piperazin-1-yl]-2-oxo-ethyl}-amide,
(19) (R)-1-(3,5-dibromo-4-hydroxy-benzyl)-2-[4-(4-methyl-piperazin-1-yl)-piperidin-1-yl]-2-oxo-ethyl 4-(2-oxo-1,2,4,5-tetrahydro-1,3-benzodiazepin-3-yl)-piperidine-1-carboxylate,
(20) (R)-1-(4-hydroxy-3,5-dimethyl-benzyl)-2-oxo-2-(4-piperazin-1-yl-piperidin-1-yl)-ethyl 4-(2-oxo-1,2,4,5-tetrahydro-1,3-benzodiazepin-3-yl)-piperidine-1-carboxylate,
(21) (R)-1-(4-hydroxy-3,5-dimethyl-benzyl)-2-[4-(1-methyl-piperidin-4-yl)-piperazin-1-yl]-2-oxo-ethyl 4-(2-oxo-1,2,4,5-tetrahydro-1,3-benzodiazepin-3-yl)-piperidine-1-carboxylate, and
(22) (S)-1-1,4′-bipiperidinyl-1′-yl-2-(3-chloro-4-hydroxy-5-trifluoromethyl-benzyl)-4-[4-(2-oxo-1,2,4,5-tetrahydro-1,3-benzodiazepin-3-yl)-piperidin-1-yl]-butan-1,4-dione,
or a physiologically acceptable salt thereof.

2. The method according to claim 1, wherein the method is practiced concomitantly with hormone replacement therapy.
